(19) United States
(12) Patent Application Publication
(10) Pub. No.: US 2014/0276041 A1
(43) Pub. Date: Sep. 18, 2014

(54) PEDIATRIC ORAL DELIVERY DEVICE AND RELATED METHOD

(71) Applicant: BEEKLEY CORPORATION, Bristol, CT (US)

(72) Inventors: Marc Hamet, Riverside, CT (US); Thomas A. Johnson, Sandy Hook, CT (US); Maeghan E. Traboulsi, Fairfax, VA (US)

(73) Assignee: BEEKLEY CORPORATION, Bristol, CT (US)

(21) Appl. No.: 13/837,334

(22) Filed: Mar. 15, 2013

(51) Int. Cl. A61M 31/00

(52) U.S. Cl. CPC A61M 31/005
USPC 600/432; 600/435

(57) ABSTRACT

An oral delivery device for delivering substance directly into a mouth of a patient includes an elongated flexible conduit coupled to a connector at a proximal end thereof for connecting to a substance source, and a nipple portion at an opposing distal end thereof for placement into a mouth of a patient. The nipple portion defines a flow aperture at a distal tip thereof. The distal end of the flexible conduit is located substantially immediately adjacent to, and in sealed fluid communication with, the flow aperture, such that substance exiting the distal end of the conduit substantially directly exits the nipple portion through the flow aperture and enters into the mouth of the patient.

### FIELD OF THE INVENTION

The present invention relates to delivery device for orally administering substance to a patient, and more particularly to a delivery device for orally administering substance to a pediatric patient.

### BACKGROUND INFORMATION

In the field of medical imaging, a patient is sometimes required to drink or ingest an imaging contrast fluid in order to improve the visibility of the patient's internal body structures in the image. In some types of medical imaging, such as, for example, computed tomography (“CT”) imaging, a patient is required to ingest the contrast fluid prior to the imaging procedure. In other types of medical imaging, such as, for example, fluoroscopy, a patient is required to ingest the contrast fluid during the imaging, in order to show real time moving images of the patient's internal body structures. Accordingly, the patient is within the radiation field of the imaging device while drinking the fluid.

When the patient is a pediatric patient, such as, for example, an infant or toddler, getting the patient to drink the contrast fluid involves certain challenges not present with a typical adult or older child. Getting such a patient to drink the contrast fluid at the particular time needed, e.g., during the imaging procedure, can be especially so. The child may not have sufficiently developed mental or language skills to understand what it needs to do, or the physical development to comply. Even if the patient does, it may not comply. At that exact time, for example, the infant may not be thirsty and/or may dislike the taste of the contrast fluid and refuse to drink it. Additionally, the patient may be frightened by the surrounding foreign environment, sounds, and individuals, and thus refuse to drink.

One approach to delivering the fluid includes having a practitioner, e.g., a nurse or technician, attempt to feed the pediatric patient the required amount of contrast fluid during the procedure via a standard baby bottle or “sippy cup”, from which an infant or child is accustomed to drinking. This still requires the patient to ingest the fluid, e.g., suck on the bottle.

This approach also has several medical drawbacks. One drawback associated with such an approach is that the volume of radio-dense contrast fluid within the bottle, e.g., barium-based, is relatively large and concentrated within the field of radiation, and therefore may obscure the patient's tissues in the image, as shown by bottle BB in FIG. 1.

Another drawback associated with such an approach is that a practitioner must often hold the bottle for the pediatric patient, and therefore must stand very close to the pediatric patient. Therefore, the practitioner must stand substantially within or near the field of radiation and thus is exposed to high levels of hazardous radiation. In particular, the practitioner's hand(s), holding the bottle (e.g., fingers F in FIG. 1), may be subjected to radiation induced injuries, especially over time. Further, the patient may be frightened by the practitioner attempting to feed him/her, and thus refuse to drink the contrast fluid.

Yet another drawback associated with such an approach is encountered with the usage of automatic self-adjusting imaging machines, such as some fluoroscopy machines. Such machines are configured to automatically adjust the radiation dose applied to the radiation field in order to achieve optimal and constant imaging quality. One factor that causes an increase in the radiation dose emitted is the detection of increased radiographic density within the radiation field. Thus, detection of the relatively large volume of radio-dense contrast fluid in a baby bottle within the radiation field, and further, the hand(s) or other body parts of the practitioner holding the bottle (see FIG. 1), results in an increase of the emitted radiation, thereby applying more radiation to the pediatric patient, and also to the practitioner holding the bottle. Increased radiation subjects both the patient and practitioner to a greater potential for radiation induced injuries.

Another approach to deliver the fluid includes having a practitioner place a syringe in the patient's mouth and inject or dispense the contrast in the syringe directly into the mouth. This approach attempts to alleviate the difficulty of a patient who refuses to ingest, and ensure contrast is ingested at the proper time, e.g., during the imaging procedure. Similarly as discussed above, however, one drawback associated with such an approach is that the practitioner must either stand within or near the field of radiation to hold and operate the syringe, thereby still being exposed to relatively high levels of radiation and potentially frightening the pediatric patient. Further, radio-dense fluid in the syringe, and potentially the technician's body parts, can obscure the patient image. Accordingly, self-adjusting imaging machines can increase the radiation levels to compensate.

### SUMMARY OF THE INVENTION

It is an object of the present invention to overcome one or more of the above-described drawbacks and/or disadvantages of the prior art. In accordance with a first aspect, an oral delivery device for delivering substance directly into a mouth of a patient comprises an elongated flexible conduit coupled to a connector at a proximal end thereof for connecting to a substance source, and a nipple portion at an opposing distal end thereof for placement into a mouth of a patient. The nipple portion defines a flow aperture at a distal tip thereof. The distal end of the flexible conduit is located substantially immediately adjacent to, and in sealed fluid communication with, the flow aperture, such that substance exiting the distal end of the conduit substantially directly exits the nipple portion through the flow aperture and enters into the mouth of the patient

In some embodiments, the oral delivery device further comprises a mouth piece defining the nipple portion and including a base portion laterally extending from a base end of the nipple portion. In some such embodiments, the base portion includes a tab portion projecting therefrom for manual handling of the mouth piece.

In some embodiments, the flexible conduit defines a length within the range of about 18 inches to about 30 inches. In some such embodiments, the flexible conduit defines a length of about 24 inches. In some embodiments, the flexible conduit defines an inner diameter of about 0.150 inch. In some embodiments, the flow aperture defines a diameter greater than about 0.02 inch. In some such embodiments, the flow aperture defines a diameter within the range of about 0.05 inch to about 0.12 inch.

In some embodiments, the substance comprises medical imaging oral contrast fluid. In some such embodiments, the contrast fluid includes barium.

In some embodiments, the nipple portion substantially simulates a female nipple. In some embodiments, the patient is one of an infant or a toddler.

In accordance with another aspect, an oral delivery device for delivering substance directly into a mouth of a patient comprises first means for flowing substance therethrough coupled to second means at a proximal end thereof for fluidly connecting the first means with a substance source, and third means at an opposing distal end thereof for fluidly connecting the first means with a patient's mouth. The third means includes fourth means for dispensing substance from the third means. A distal end of the first means is located substantially immediately adjacent to, and in sealed fluid communication with, the fourth means such that substance exiting the first means substantially directly exits the third means through the fourth means and enters into the mouth of the patient.

In some embodiments, the first means is a flexible conduit, the second means is a connector, the third means is a nipple portion and the fourth means is a flow aperture at a distal tip of the nipple portion.

In accordance with another aspect, a method comprises the steps of:

(I) situating a patient into a field of radiation of a medical imaging device;

(II) placing a nipple portion of an oral delivery device into a mouth of the patient, wherein the delivery device comprises an elongated flexible conduit coupled to a connector at a proximal end thereof and with the nipple portion at an opposing distal end thereof; wherein the nipple portion defines a flow aperture at a distal tip thereof;

(III) connecting a substance source to the delivery device; and

(IV) administering the substance through the delivery device and directly into the mouth of the patient from substantially outside the field of radiation.

In some embodiments, the substance is administered from at least about 18 inches from the patient.

In some embodiments, the distal end of the flexible conduit is located substantially immediately adjacent to, and in sealed fluid communication with, the flow aperture, and the administering step comprises injecting the substance from the source and through the connector wherein upon exiting the distal end of the conduit, the substance substantially directly exits the nipple portion through the flow aperture and enters into the mouth of the patient.

Other objects and advantages of the present invention, and/or of the currently preferred embodiments thereof, will become more readily apparent in view of the following detailed description of currently preferred embodiments and accompanying drawings.

### BRIEF DESCRIPTION OF THE DRAWINGS

FIG. 1 is a radiographic image showing obscuring of the patient image by a baby bottle utilized for ingestion of oral contrast fluid by a pediatric patient from a baby bottle held by a practitioner;

FIG. 2 is a an oral delivery device during operation;

FIG. 2a is a side view of the delivery device of FIG. 2.

FIG. 3 is an enlarged partial cross-section view of the oral delivery device of FIG. 2;

FIG. 4 is a radiographic image showing minimal obscuring in the image when utilizing the oral delivery device of FIG. 2 to deliver oral contrast fluid to a pediatric patient;

FIG. 5 is another embodiment of an oral delivery device;

FIG. 6 is an enlarged partial view of the distal end of the oral delivery device of FIG. 5; and

FIG. 7 is an enlarged cross-sectional partial view of the distal end of the oral delivery device of FIG. 5 taken along section line A-A in FIG. 5.

### DETAILED DESCRIPTION OF EMBODIMENTS OF THE INVENTION

In FIGS. 2-4, a device is indicated generally by the reference numeral 10. In the illustrated embodiment, the device 10 is an oral delivery device for administering substance, e.g., medical imaging contrast fluid, such as barium or iodine, to a pediatric patient, such as, for example, an infant or a toddler.

As shown in FIGS. 2 and 2a, the delivery device 10 includes an elongated flexible feeding conduit or tube 12 coupled in fluid communication between a connector 14 at a proximal end thereof and a mouth piece 16 at an opposing distal end thereof. The connector 14 is utilized to connect the proximal end of the device 10 to a substance source, such as, for example, a syringe 50 containing the required amount of contrast fluid. The mouth piece 16 is placed in the patient's mouth, so that the substance can be administered, e.g., injected, from the source, through the device 10, and exit the device 10 directly inside the patient's mouth. In the illustrated embodiment the connector 14 is a LUER connector. However, as should be understood by those of ordinary skill in the pertinent art, the connector 14 can take the form of any of numerous different connectors capable of connecting the proximal end of the tube 12 to a substance source.

In the illustrated embodiment, the feeding tube 12 is a silicone tube. However, as should be understood by those of ordinary skill in the pertinent art, the flexible tube may be made of any of numerous different flexible materials, or medical or food grade tubing, currently known or that later become known. The tube 12 is sufficiently long to allow a practitioner to administer the substance to a patient from a distance to the patient, e.g., from substantially outside a radiographic imaging field of radiation, to reduce the radiation exposure to the practitioner, and also to remove the substance source, e.g., syringe 50, from interfering with the imaging of the patient.

As shown best in FIG. 3, the mouth piece 16 includes a flexible and lateral proximal base portion or shield 18 having a flexible, elongated and substantially hollow nipple portion 20 projecting distally therefrom. The mouth piece 16 is configured to be placed into the mouth of a pediatric patient. The shield 18 prevents over-insertion or ingestion of the mouthpiece. In the illustrated embodiment, the nipple portion 20 is shaped and dimensioned to simulate a female nipple or a pacifier, in order to enhance the natural feel of the mouth piece 16 to the pediatric patient and encourage the patient to suckle the portion 20 as he/she would naturally suckle a mother's nipple or a pacifier. As should be understood by those of ordinary skill in the pertinent art, however, the nipple portion can be shaped and dimensioned in any of numerous other configurations that would be comfortable for a pediatric patient to receive in his/her mouth. The distal tip 21 of the nipple portion 20 defines a flow aperture 22 therein for the passage of substance therethrough and directly into the mouth of the pediatric patient. In the illustrated embodiment, the flow aperture 22 is approximately centrally located in the nipple portion 16. However, in other embodiments the flow aperture is not centrally located.

As should be understood by those of ordinary skill in the pertinent art, resistance against flow through the device 10, i.e., back-pressure, is caused, in part, by a combination of factors including the length of the tube as well as the diameter of the tube and internal friction forces within the tube, and the size of the aperture 22 at the distal end of the device 10. The back-pressure applied by the tube 12 against the flow therethrough is manifested in the amount of force required by a practitioner to administer the substance through the device 10, e.g., the amount of force required to depress a syringe plunger within a syringe 50 connected to the tube 12 to administer the substance therethrough. Additionally, the flow resistance limits the amount of flow through and out of the device. This can have implications for procedures, e.g., fluoroscopy, where a certain flow rate out of the device and into the patient is desired for diagnosis. If the flow restriction is too high, the flow rate (ingestion rate) may be too low. Thus, as the length of the tube increases, the diameter of the tube decreases, and the size of the aperture decreases, a practitioner needs to apply a greater force when advancing the syringe plunger through the syringe 50 in order to administer the substance through the tube 12, and regardless may not be able to deliver a sufficient volumetric rate of fluid. The tube 12 is therefore sufficiently short and of sufficient diameter, and the aperture is of sufficient size, to mitigate the presence of an impractical or undue amount of back pressure and flow restriction, which would make it substantially difficult for a practitioner to administer the substance through the device 10, and at a sufficient flow rate.

In some exemplary embodiments, the tube 12 defines a length within the range of about 12 inches to about 60 inches. In some such embodiments, the tube 12 defines a length within the range of about 18 inches to about 36 inches. In yet some such embodiments, the tube 12 is about 24 inches long. In some exemplary embodiments, the tube 12 defines an inner diameter D within the range of about 0.125 inch to about 0.175 inch. In some such embodiments, the tube 12 defines an inner diameter D of approximately 0.150 inch. In some exemplary embodiments, the aperture 22 defines a diameter D2 greater than about 0.02 inch. In some such embodiments, the aperture 22 defines a diameter D2 greater than about 0.03 inch. In yet some such embodiments, the aperture 22 defines a diameter D2 greater than about 0.05 inch, or within the range of about 0.05 inch to about 0.12 inch. The inventors have found that the combination of tube length and inner diameter, as well as flow aperture size, within the above-described ranges, mitigates the amount of back-pressure that must be overcome by a practitioner in order to administer the substance and allow sufficient flow rate, while also allowing a practitioner to administer the substance to the patient from a distance to the patient and/or the field of radiation so as to reduce practitioner radiation exposure. The flow aperture size within the above-described ranges, in combination with the connection of the syringe 50 to the tube 12, also assists in mitigating unintended leaking of substance from the aperture 22 without active advancement of the substance by the practitioner, or suckling of the mouth piece 16 by the pediatric patient.

In the illustrated embodiment, the substantially hollow nipple portion 20 includes a substantially cylindrical sleeve portion 24 therein, sealingly surrounding the flow aperture 22 at the distal tip 21 of the nipple portion 20, and projecting proximally therefrom. The distal/outlet end of the tube 12 is sealingly received within the sleeve 24 and creates a substantially fluid tight seal therebetween. The outlet end of the tube 12 extends within the sleeve 24 to a position substantially immediately adjacent to, and in fluid communication with, the flow aperture 22. In some embodiments, the outlet end of the nipple is located within 3/16″ of the aperture 22, such as within 2/16″ or within 1/16″.

As should be understood by those of ordinary skill in the pertinent art, the tube 12 can be sealingly engaged with the sleeve 24 in any of numerous different manners. For example, and without limitation, the tube 12 may be bonded to the sleeve 24, e.g., adhesively or heat-bonded, co-molded with the sleeve, or form an interference, press or force fit with the sleeve. As also should be understood by those of ordinary skill in the pertinent art, the distal end of the tube 12 may alternatively be directly attached to the distal tip 21 of the nipple portion 20, surrounding the flow aperture 22, to place the tube in direct fluid communication therewith.

In some embodiments, the base portion 18 of the mouth piece 16 also includes a tab 26. In the illustrated embodiment, the tab 26 projects substantially proximally from a perimeter portion of the base portion 18 and is formed integral therewith. Alternatively, the tab 26 can be separately formed and attached to the base portion 18 and can project from the base portion in another direction. The tab 26 can be utilized for handling the base portion 18 when inserting or removing the nipple portion 20 into or out of the mouth of a pediatric patient.

In use, the device 10 can be utilized to administer substance, such as oral contrast fluid, to a pediatric patient, prior to or during an imaging procedure. In an exemplary embodiment, a syringe 50 pre-filled with the desired volume of contrast fluid can be connected to the tube 12 via the connector 14. The nipple portion 20 of the mouth piece 16 is placed into the mouth of the pediatric patient to allow the patient to begin suckling the nipple portion. In some embodiments, and in preparation for administering the fluid, the practitioner can actuate the syringe plunger to advance the oral contrast partially through the tubing toward the nipple portion 20 prior to placing the portion 20 into the patient's mouth. Alternatively, the practitioner can place the nipple portion 20 into the patient's mouth first.

Once the nipple portion 20 is in the patient's mouth, the practitioner will advance the desired volume of contrast fluid through the tube 12 and out of the flow aperture 22 at the distal end thereof, to administer the fluid directly into the patient's mouth for ingestion. As the patient suckles on the nipple portion 20 as he/she would on a mother's nipple or feeding bottle, the fluid administered into the patient's mouth is swallowed by the patient. However, because the dispensing action by the practitioner injects the fluid into the mouth even if the patient does not suckle, it is not necessary for the patient to suckle to withdraw substance from the delivery device. Further, once administered into the patient's mouth, the patient's natural instincts are to swallow the substance. In addition, as the tube 12 does not extend past the flow aperture 22, i.e., out of the nipple portion 20, the tube does not introduce a foreign or unfamiliar object or shape into the mouth, which can cause the patient discomfort or resist administration of the substance.

Due to the above-described configuration of the device 10, particularly the location in and connection of the tube 12 to the nipple portion, relative to the flow aperture, the fluid flows substantially directly into the patient's mouth, and very little, if any, fluid accumulates within the nipple portion 20 (outside of the tube 12). This ensures that substantially all of the desired volume or dose is administered into the patient's mouth (as opposed to part of it remaining in the nipple). It also minimizes obscuring of patient tissues in the radiographic image by fluid in the device.

Where a medical image is performed that requires drinking of the contrast fluid during the imaging procedure, such as, for example, during fluoroscopy, the length of the tube 12 enables the practitioner to administer the contrast fluid to the patient while standing away from the patient, e.g., substantially outside of the field of radiation. Upon completion of the imaging procedure, the nipple portion 20 can be removed from the patient's mouth and discarded.

One advantage of the invention is that substance advanced through the feeding tube 12 from the connector end thereof exits the feeding tube 12 at, and substantially directly through, the flow aperture 22. As the outlet end of the tube 12 is located substantially immediately adjacent the flow aperture 22, and the sleeve 24 seals the space between the distal end of the tube 12 and the flow aperture 22, the substance is substantially prevented from exiting the tube 12 and accumulating elsewhere within the nipple portion 20. Rather, the substance exiting the tube 12 substantially directly exits out of the nipple portion 20 through the aperture 22 and into the patient's mouth. This is advantageous for several reasons. First, though suckling can adequately draw substance out of the device, active suckling of the substance out of the nipple portion 20 by the patient is not directly required for delivery of the substance. Rather, while suckling of the nipple portion 20 assists in drawing the substance from the device 10, it is the practitioner's active administering of the substance through the device 10, e.g., by advancing the plunger of the syringe 50, that primarily delivers the substance into the patient's mouth. Thus, rejection of the substance by the patient is mitigated.

Further, as suckling of the nipple portion 20 mimics the natural feeding process, the likelihood that the patient will accept and swallow the substance is increased. Yet further, because the patient substantially does not contact the tube 12 while suckling the nipple portion 20, the natural feeling of the nipple portion 20 is substantially preserved, thereby encouraging feeding. Alternatively for example, projection of the outlet end of the tube 12 out of, and beyond, the nipple portion 20 could interfere with the natural suckling motion of the patient and thus negatively affect feeding.

Another advantage associated with the above-described delivery device 10 is that the substance within the nipple portion 20 resides within the tube 12, and substantially does not further accumulate elsewhere within the nipple portion 20. Thus, when delivering a radio-dense substance to a patient, e.g., oral imaging contrast, during a radiographic imaging procedure, the amount of radio-dense substance located within the field of radiation is minimized, as shown in FIG. 4, thereby minimizing obscuring of the patient in the radiographic image. Moreover, with the use of automatic self-adjusting radiographic imaging machines, reduction of radio-dense contrast fluid within the field of radiation results in reduced radiation emission by the machine. This mitigates radiation exposure by the patient and practitioner.

Testing performed has shown a decrease in radiation emitted by a self-adjusting imaging machine when utilizing the delivery device 10, resulting in approximately a 25% reduction in radiation dose (32 mR to 24 mR) to a pediatric patient compared to when using the above-described traditional baby bottle technique. Even if a bottle that is half full is used, that would still provide a reduction of about half of the above, i.e., 12% or 28 mR.

Yet another advantage associated with the above-described delivery device 10 is the capability for a practitioner to administer substance to the patient at a distance from the patient and even outside the field of radiation, due to the length of the tube 12, thereby reducing the practitioner's exposure to radiation. Based on a general rule of 0.1% of the useful beam dose at one meter (36 inches) from the primary beam, it is estimated that, in view of the inverse square rule in which the reduction in exposure at one distance as compared to another is the square of the ratio of the distances, doubling the distance of the practitioner's body from the primary beam from about 12 inches (e.g., using a traditional baby bottle) to about 24 inches would result in approximately a 12% (4 times) reduction in radiation exposure to the body of a practitioner (e.g., scatter radiation) administering the substance to a patient during a radiographic imaging procedure.

(24″/12″)²=4

Testing has also shown approximately a 99% reduction in radiation dose (32 mR to 0.07 mR or 500 times) to the hands of a practitioner compared to when using the traditional baby bottle technique. Specifically, as discussed above, replacing a half-full bottle held by the practitioner's hand in the radiation field with the invention reduces the radiation dose about from 32 mR to 28 mR. Based on 0.1% of the useful beam dose at one meter (3 feet) from the primary beam, the inverse square correction for the hand now being 2 feet from the beam edge is as follows:

28 mR*0.1%*(3′/2′)²=0.07 mR

Thus, the invention provides significant benefit to practitioners, who would enjoy substantially reduced cumulative radiation exposure over time, especially to the hand or fingers.

In FIGS. 5-7, another delivery device is indicated generally by the reference numeral 110. The device 110 is substantially similar to the device 10 described above in connection with FIGS. 2-4, and therefore like reference numerals preceded by the numeral “1” are used to indicate like elements.

As shown in FIG. 5, the connector 114 at the proximal end of the delivery device 110 is a tapered or cone-like fitting. In the illustrated embodiment, as shown best in FIG. 6, the connector 114 includes a knurled surface or barbs 114a for gripping and/or sealing engagement with the proximal end of the tube 112. The connector 114 is adapted to receive therein a corresponding tapered or cone-like tip 154 of a substance source 150, such as, for example, a syringe containing the required amount of contrast fluid.

At the opposing distal end of the device 110 (see FIG. 7), the nipple portion 120 of the mouth piece 116 is not substantially hollow. Rather the nipple portion 120 is substantially solid relative to the nipple portion 20 and includes an approximately central channel 124 therein extending from the hollow cavity 116a in the mouth piece 116 through the nipple portion 120. In other embodiments the channel 124 is not centrally located. The channel 124 defines the flow aperture 122 at the distal end thereof. As shown in FIG. 7, the distal/outlet end of the tube 112 is fittingly received within the channel 124 and extends within the channel to a position substantially immediately adjacent to the aperture 122. The tube 112 and the channel 124 create a fluid tight seal therebetween. As seen in FIG. 7, the channel 124 has a wider portion 124a that receives that tube 112, and a narrower portion 124b that extends from the wider portion 124a to the flow aperture 122. The junction of the wider portion 124a and the narrower portion 124b forms a seat 124c that receives the distal end of the tube 112. The diameter D2 of the narrower portion 124b is approximately the same as the inner diameter D of the tube 112 so as to mitigate flow disruption when contrast flows from the tube 122 into the channel 124.

In some other embodiments, for example, the tube 112 creates a dimensional interference with the channel 124, resulting in an interference fit therebetween. However, similarly to as described with respect to the device 10, the tube 112 can be sealingly engaged with the channel 124 in any of numerous different manners. Substance advanced through the feeding tube 112 from the connector end thereof exits the feeding tube 112 at, and substantially directly through, the flow aperture 122.

It should be understood that the term “about,” “approximately” and like terms used herein when referring to a dimension or characteristic of the delivery device of the invention indicate that the described dimension/characteristic is not a strict boundary or parameter and does not exclude variations therefrom that are functionally similar. At a minimum, such references that include a numerical parameter would include variations that, using mathematical and industrial principles accepted in the art (e.g., rounding, measurement or other systematic errors, manufacturing tolerances, etc.), would not vary the least significant digit.

As may be recognized by those of ordinary skill in the pertinent art based on the teachings herein, numerous changes and modifications may be made to the above-described and other embodiments of the present invention without departing from its scope as defined in the claims. For example, changes to the material forming the device, the dimensions of the feeding tube and/or the flow aperture or the configuration of mouth piece can be made. Accordingly, this detailed description of embodiments is to be taken in an illustrative, as opposed to a limiting sense.

What is claimed is:

1. An oral delivery device for delivering substance directly into a mouth of a patient comprising:
an elongated flexible conduit coupled to a connector at a proximal end thereof for connecting to a substance source, and a nipple portion at an opposing distal end thereof for placement into a mouth of a patient;
wherein the nipple portion defines a flow aperture at a distal tip thereof; and
wherein the distal end of the flexible conduit is located substantially immediately adjacent to, and in sealed fluid communication with, the flow aperture, such that substance exiting the distal end of the conduit substantially directly exits the nipple portion through the flow aperture and enters into the mouth of the patient.

2. An oral delivery device as defined in claim 1, further comprising a mouth piece defining the nipple portion and including a base portion laterally extending from a base end of the nipple portion.

3. An oral delivery device as defined in claim 3, wherein the base portion includes a tab portion projecting therefrom for manual handling of the mouth piece.

4. An oral delivery device as defined in claim 1, wherein the flexible conduit defines a length within the range of about 18 inches to about 30 inches.

5. An oral delivery device as defined in claim 4, wherein the flexible conduit defines a length of about 24 inches.

6. An oral delivery device as defined in claim 1, wherein the flexible conduit defines an inner diameter of about 0.150 inch.

7. An oral delivery device as defined in claim 1, wherein the flow aperture defines a diameter greater than about 0.02 inch.

8. An oral delivery device as defined in claim 7, wherein the flow aperture defines a diameter within the range of about 0.05 inch to about 0.12 inch.

9. An oral delivery device as defined in claim 1, wherein the connector comprises a LUER connector.

10. An oral delivery device as defined in claim 1, wherein the substance comprises medical imaging oral contrast fluid.

11. An oral delivery device as defined in claim 10, wherein the contrast fluid includes barium.

12. An oral delivery device as defined in claim 1, wherein the nipple portion substantially simulates a female nipple.

13. An oral delivery device as defined in claim 1, wherein the patient is one of an infant or a toddler.

14. An oral delivery device as defined in claim 1, wherein the substance source is a syringe pre-filled with a desired volume of the substance.

15. An oral delivery device for delivering substance directly into a mouth of a patient comprising:
first means for flowing substance therethrough coupled to second means at a proximal end thereof for fluidly connecting the first means with a substance source, and third means at an opposing distal end thereof for fluidly connecting the first means with a patient's mouth;
wherein the third means includes fourth means for dispensing substance from the third means; and
wherein a distal end of the first means is located substantially immediately adjacent to, and in sealed fluid communication with, the fourth means such that substance exiting the first means substantially directly exits the third means through the fourth means and enters into the mouth of the patient.

16. An oral delivery device as defined in claim 15, wherein the first means is a flexible conduit, the second means is a connector, the third means is a nipple portion and the fourth means is a flow aperture at a distal tip of the nipple portion.

17. An oral delivery device as defined in claim 15, wherein the patient is one of an infant or a toddler.

18. A method comprising:
situating a patient into a field of radiation of a medical imaging device;
placing a nipple portion of an oral delivery device into a mouth of the patient, wherein the delivery device comprises an elongated flexible conduit coupled to a connector at a proximal end thereof and with the nipple portion at an opposing distal end thereof; wherein the nipple portion defines a flow aperture at a distal tip thereof;
connecting a substance source to the delivery device; and
administering the substance through the delivery device and directly into the mouth of the patient from substantially outside the field of radiation.

19. A method as defined in claim 18, wherein the substance is administered from at least about 18 inches from the patient.

20. A method as defined in claim 18, wherein the connecting step comprises connecting the substance source to the connector of the delivery device.

21. A method as defined in claim 18, wherein the distal end of the flexible conduit is located substantially immediately adjacent to, and in sealed fluid communication with, the flow aperture, and the administering step comprises injecting the substance from the source and through the connector wherein upon exiting the distal end of the conduit, the substance substantially directly exits the nipple portion through the flow aperture and enters into the mouth of the patient.
